(19) United States
(12) Patent Application Publication
(10) Pub. No.: US 2022/0220099 A1
(43) Pub. Date: Jul. 14, 2022

(54) BENZAMIDE COMPOUND, AND HERBICIDE

(71) Applicant: Nippon Soda Co., Ltd., Chiyoda-ku, Tokyo (JP)

(72) Inventors: Ken MIHARA, Kanagawa (JP); Yusuke FUKUSHIMA, Shizuoka (JP); Yukina TAKI, Kanagawa (JP); Yoji IKEDA, Kanagawa (JP); Kazushige KATO, Kanagawa (JP); Jun INAGAKI, Kanagawa (JP)

(73) Assignee: Nippon Soda Co., Ltd., Chiyoda-ku, Tokyo (JP)

(21) Appl. No.: 17/615,667

(22) PCT Filed: Jun. 1, 2020
(86) PCT No.: PCT/JP2020/021572

§ 371 Date: Dec. 1, 2021

(30) Foreign Application Priority Data
Jun. 4, 2019 (JP) 2019-104650

(51) Int. Cl. C07D 409/12; C07D 413/12; A01N 43/653; A01N 43/82; A01N 43/713; A01P 13/00

(52) U.S. Cl. CPC C07D 409/12; C07D 413/12; A01P 13/00; A01N 43/82; A01N 43/713; A01N 43/653

(57) ABSTRACT

It is to provide a novel benzamide compound useful as an active ingredient of a herbicide, the benzamide compound exhibiting a definite weed control effect even in a small amount, having low phytotoxicity to crops, and having high safety in environments, and a herbicide. A compound of the present invention is a compound of formula (II) or a salt thereof. In formula (II), Het is a substituted or unsubstituted five-membered heteroaryl group; R¹ is a substituted or unsubstituted C1-6 alkyl group, a substituted or unsubstituted C1-6 alkoxy group, or the like; R² is a hydrogen atom, a substituted or unsubstituted C1-6 alkyl group, or the like; X¹ is an oxygen atom or a sulfonyl group; X² is an oxygen atom, a sulfenyl group, or the like; m is 0 or 1, n is 0 or 1, and the sum of m and n is 1 or 2; A is a substituted or unsubstituted C2-4 alkylene group, a substituted or unsubstituted C2-3 alkenylene group, or the like; R³ is a hydrogen atom, a substituted or unsubstituted C1-6 alkyl group, or a halogeno group; R⁴ is a hydrogen atom, a substituted or unsubstituted C1-6 alkyl group, or a halogeno group; and Z is an oxygen atom or a sulfur atom.

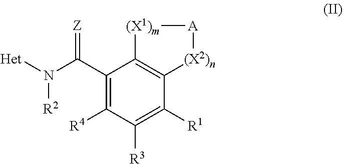

### TECHNICAL FIELD

The present invention relates to a novel benzamide compound and a herbicide comprising the novel benzamide compound as an active ingredient.

The present application claims priority to Japanese Patent Application No. 2019-104650 filed on Jun. 4, 2019, which is herein incorporated by reference.

### BACKGROUND ART

In cultivation of agricultural and horticultural crops, a herbicide is used in some cases for the purpose of controlling weeds. Various compounds have so far been proposed as an active ingredient of a herbicide. For example, patent document 1 discloses a bicyclic benzamide compound of formula (A).

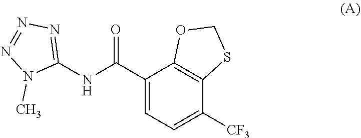 [SMILES: Cn1nnnc1NC(=O)c1ccc(C(F)(F)F)c2c1OCS2]

### PRIOR ART DOCUMENTS

### Patent Documents

Patent Document 1: WO2019/096884

### SUMMARY OF THE INVENTION

### Object to be Solved by the Invention

A herbicide is required not only to have an excellent efficacy in weed control activity, but also to have low phytotoxicity to crops, to be hard to remain in an environment, and not to pollute an environment, and the like. An object of the present invention is to provide a novel benzamide compound useful as an active ingredient of a herbicide, that has a definite weed control effect even in a small dose, has low phytotoxicity to crops, and has high an environmental safety, and a herbicide.

### Means to Solve the Object

Studies have been conducted in order to achieve the object, and as a result, the present invention including the following embodiments have been completed.

[1] A compound of formula (II) or a salt thereof.

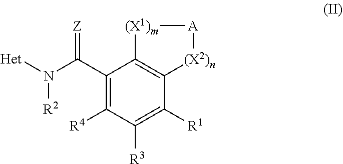

wherein

Het is a substituted or unsubstituted five-membered heteroaryl group;

R¹ is a substituted or unsubstituted C1-6 alkyl group, a substituted or unsubstituted C1-6 alkoxy group, a substituted or unsubstituted C1-6 alkylthio group, a substituted or unsubstituted C6-10 aryl group, a substituted or unsubstituted five- to six-membered heteroaryl group, or a halogeno group;

R² is a hydrogen atom, a substituted or unsubstituted C1-6 alkyl group, a substituted or unsubstituted C1-6 alkylcarbonyl group, a substituted or unsubstituted C1-6 alkoxycarbonyl group, or a substituted or unsubstituted C6-10 arylcarbonyl group;

X¹ is an oxygen atom or a sulfonyl group;

X² is an oxygen atom, a sulfenyl group, a sulfinyl group, a sulfonyl group, a group represented by —S(═NR)—, or a group represented by —S(═O) (═NR)—, wherein

each R independently is a hydrogen atom, a C1-6 alkyl group, a substituted or unsubstituted C1-6 alkylcarbonyl group, a substituted or unsubstituted C6-10 aryl group, or a cyano group;

m is 0 or 1, n is 0 or 1, and the sum of m and n is 1 or 2;

A is a substituted or unsubstituted C2-4 alkylene group, a substituted or unsubstituted C2-3 alkenylene group, or a substituted or unsubstituted C1-2 alkyleneoxy C1-C2 alkylene group;

R³ is a hydrogen atom, a substituted or unsubstituted C1-6 alkyl group, or a halogeno group;

R⁴ is a hydrogen atom, a substituted or unsubstituted C1-6 alkyl group, or a halogeno group; and

Z is an oxygen atom or a sulfur atom.

[2] The compound or a salt thereof according to [1], wherein R³ is a hydrogen atom, R⁴ is a hydrogen atom, and Z is an oxygen atom.
[3] The compound or a salt thereof according to [1], wherein m is 0, and n is 1.
[4] A herbicide comprising as an active ingredient at least one compound or salt selected from the group consisting of the compound and a salt thereof according to any one of [1] to [3].

### Effect of the Invention

A benzamide compound of the present invention exhibits a definite weed control effect even in a small dose, has low phytotoxicity to crops, and has high an environmental safety, and is therefore useful as an active ingredient of a herbicide. A herbicide of the present invention can be used safely for controlling weeds in cultivation of agricultural and horticultural crops.

### MODE OF CARRYING OUT THE INVENTION

A benzamide compound of the present invention is a compound of formula (II) (sometimes referred to as compound (II)) and a salt of the compound (II). In the benzamide compound of the present invention, hydrates, various solvated substances, crystal polymorphism, and the like are included. Stereoisomers and tautomers based on asymmetric carbon, a double bond, and the like can exist in the benzamide compound of the present invention. All of such isomers and mixtures thereof are included in the technical scope of the present invention.

The term “unsubstituted” which is used in the present specification means being only a group to be a mother nucleus. Only a name of a group to be a mother nucleus, when described without describing “substituted,” means to be “unsubstituted” unless otherwise noted.

On the other hand, the term “substituted” means that any one of hydrogen atoms of a group to be a mother nucleus is substituted with a group (substituent) having a structure which is the same or different from that of the mother nucleus. Accordingly, the “substituent” is another group bonded to a group to be a mother nucleus. The number of substituents may be one, or two or more. Two or more substituents may be the same or different.

A term such as “C1-6” shows that the number of carbon atoms of a group to be a mother nucleus is 1 to 6, or the like. The number of carbon atoms in a substituent is not included in this number of carbon atoms. For example, a butyl group having an ethoxy group as a substituent is classified into a C2 alkoxy C4 alkyl group.

The “substituent” is not particularly limited as long as it is chemically permissible and has the effects of the present invention. Examples of the group that can be a “substituent” include the followings.

A C1-6 alkyl group, such as a methyl group, an ethyl group, a n-propyl group, an i-propyl group, a n-butyl group, a s-butyl group, an i-butyl group, a t-butyl group, a n-pentyl group, and a n-hexyl group;

a C2-6 alkenyl group, such as a vinyl group, a 1-propenyl group, a 2-propenyl group (allyl group), a 1-butenyl group, a 2-butenyl group, a 3-butenyl group, a 1-methyl-2-propenyl group, and a 2-methyl-2-propenyl group;

a C2-6 alkynyl group, such as an ethynyl group, a 1-propynyl group, a 2-propynyl group, a 1-butynyl group, a 2-butynyl group, a 3-butynyl group, and a 1-methyl-2-propynyl group;

a C3-8 cycloalkyl group, such as a cyclopropyl group, a cyclobutyl group, a cyclopentyl group, a cyclohexyl group, and a cubanyl group;

a C6-10 aryl group, such as a phenyl group and a naphthyl group;

a C6-10 aryl C1-6 alkyl group, such as a benzyl group and a phenethyl group;

a three- to six-membered heterocyclyl group;

a three- to six-membered heterocyclyl C1-6 alkyl group;

a hydroxy group;

a C1-6 alkoxy group, such as a methoxy group, an ethoxy group, a n-propoxy group, an i-propoxy group, a n-butoxy group, a s-butoxy group, an i-butoxy group, and a t-butoxy group;

a C2-6 alkenyloxy group, such as a vinyloxy group, an allyloxy group, a propenyloxy group, and a butenyloxy group;

a C2-6 alkynyloxy group, such as an ethynyloxy group and a propargyloxy group;

a C6-10 aryloxy group, such as a phenoxy group and a naphthoxy group;

a C6-10 aryl C1-6 alkoxy group, such as a benzyloxy group and a phenethyloxy group;

a five- to six-membered heteroaryloxy group, such as a thiazolyloxy group and a pyridyloxy group;

a five- to six-membered heteroaryl C1-6 alkyloxy group, such as a thiazolylmethyloxy group and a pyridylmethyloxy group;

a formyl group;

a C1-6 alkylcarbonyl group, such as an acethyl group and a propionyl group;

a formyloxy group;

a C1-6 alkylcarbonyloxy group, such as an acetyloxy group and a propionyloxy group;

a C6-10 arylcarbonyl group, such as a benzoyl group;

a C1-6 alkoxycarbonyl group, such as a methoxycarbonyl group, an ethoxycarbonyl group, a n-propoxycarbonyl group, an i-propoxycarbonyl group, a n-butoxycarbonyl group, and a t-butoxycarbonyl group;

a C1-6 alkoxycarbonyloxy group, such as a methoxycarbonyloxy group, an ethoxycarbonyloxy group, a n-propoxycarbonyloxy group, an i-propoxycarbonyloxy group, a n-butoxycarbonyloxy group, and a t-butoxycarbonyloxy group;

a carboxyl group;

a halogeno group, such as a fluoro group, a chloro group, a bromo group, and an iodo group;

a C1-6 haloalkyl group, such as a chloromethyl group, a chloroethyl group, a trifluoromethyl group, a 1,2-dichloro-n-propyl group, and a 1-fluoro-n-butyl group;

a C2-6 haloalkenyl group, such as a 2-chloro-1-propenyl group and a 2-fluoro-1-butenyl group;

a C2-6 haloalkynyl group, such as a 4,4-dichloro-1-butynyl group, a 4-fluoro-1-pentynyl group, and a 5-bromo-2-pentynyl group;

a C1-6 haloalkoxy group, such as a trifluoromethoxy group, a 2-chloro-n-propoxy group, and a 2,3-dichlorobutoxy group;

a C2-6 haloalkenyloxy group, such as a 2-chloropropenyloxy group and a 3-bromobutenyloxy group;

a C1-6 haloalkylcarbonyl group, such as a chloroacetyl group, a trifluoroacetyl group, and a trichioroacetyl group;

an amino group;

a C1-6 alkyl-substituted amino group, such as a methylamino group, a dimethylamino group, and a diethylamino group;

a C6-10 arylamino group, such as an anilino group and a naphthylamino group;

a C6-10 aryl C1-6 alkylamino group, such as a benzylamino group and a phenethylamino group;

a formylamino group;

a C1-6 alkylcarbonylamino group, such as an acetylamino group, a propanoylamino group, a butylylamino group, and an i-propylcarbonylamino group;

a C1-6 alkoxycarbonylamino group, such as a methoxycarbonylamino group, an ethoxycarbonylamino group, a n-propoxycarbonylamino group, and an i-propoxycarbonylamino group;

an unsubstituted or substituted aminocarbonyl group, such as a carbonyl group, a dimethylaminocarbonyl group, a phenylaminocarbonyl group, and an N-phenyl-N-methylaminocarbonyl group;

an imino C1-6 alkyl group, such as an iminomethyl group, a (1-imino)ethyl group, and a (1-imino)-n-propyl group;

a substituted or unsubstituted N-hydroxyimino C1-6 alkyl group, such as an N-hydroxy-iminomethyl group, a (1-(N-hydroxy)-imino)ethyl group, a (1-(N-hydroxy)-imino)propyl group, an N-methoxy-iminomethyl group, and a (1-(N-methoxy)-imino)ethyl group;

an aminocarbonyloxy group;

a C1-6 alkyl-substituted aminocarbonyloxy group, such as an ethylaminocarbonyloxy group and a dimethylaminocarbonyloxy group;

a mercapto group;

a C1-6 alkylthio group, such as a methylthio group, an ethylthio group, a n-propylthio group, an i-propylthio group, a n-butylthio group, an i-butylthio group, a s-butylthio group, and a t-butylthio group;

a C1-6 haloalkylthio group, such as a trifluoromethylthio group and a 2,2,2-trifluoroethylthio group;

a C6-10 arylthio group, such as a phenylthio group and a naphthylthio group;

a five- to six-membered heteroarylthio group, such as a thiazolylthio group and a pyridylthio group;

a C1-6 alkylsulfinyl group, such as a methylsulfinyl group, an ethylsulfinyl group, and a t-butylsulfinyl group;

a C1-6 haloalkylsulfinyl group, such as a trifluoromethylsulfinyl group and a 2,2,2-trifluoroethylsulfinyl group;

a C6-10 arylsulfinyl group, such as a phenylsulfinyl group;

a five- to six-membered heteroarylsulfinyl group, such as a thiazolylsulfinyl group and a pyridylsulfinyl group;

a C1-6 alkylsulfonyl group, such as a methylsulfonyl group, an ethylsulfonyl group, and a t-butylsulfonyl group;

a C1-6 haloalkylsulfonyl group, such as a trifluoromethylsulfonyl group and a 2,2,2-trifluoroethylsulfonyl group;

a C6-10 arylsulfonyl group, such as a phenylsulfonyl group;

a five- to six-membered heteroarylsulfonyl group, such as a thiazolylsulfonyl group and a pyridylsulfonyl group;

a C1-6 alkylsufonyloxy group, such as a methylsulfonyloxy group, an ethylsulfonyloxy group, and a t-butylsulfonyloxy group;

a C1-6 haloalkylsulfonyloxy group, such as a trifluoromethylsulfonyloxy group and a 2,2,2-trifluoroethylsulfonyloxy group;

a tri C1-6 alkyl-substituted silyl group, such as a trimethylsilyl group, a triethylsilyl group, and a t-butyldimethylsilyl group;

a tri C6-10 aryl-substituted silyl group, such as a triphenylsilyl group;

a pentafluorosulfanyl group;

a cyano group; and a nitro group.

In addition, in these “substituents”, any one of the hydrogen atoms in the substituent may be substituted with a group having a different structure. Examples of the “substituent” in that case include a C1-6 alkyl group, a C1-6 haloalkyl group, a C1-6 alkoxy group, a C1-6 haloalkoxy group, a halogeno group, a cyano group, and a nitro group.

In addition, the “three- to six-membered heterocyclyl group” contains one to four heteroatoms selected from the group consisting of a nitrogen atom, an oxygen atom, and a sulfur atom as constituent atoms of the ring. The heterocyclyl group may be monocyclic or polycyclic. When at least one of the rings in the polycyclic heterocyclyl group is a heteroring, the other rings may be any of a saturated aliphatic ring, an unsaturated aliphatic ring, or an aromatic ring. Examples of the “three- to six-membered heterocyclyl group” include a three- to six-membered saturated heterocyclyl group, a five- to six-membered heteroaryl group, and a five- to six-membered partially unsaturated heterocyclyl group.

Examples of the three- to six-membered saturated heterocyclyl group include an aziridinyl group, an epoxy group, a pyrrolidinyl group, a tetrahydrofuranyl group, a thiazolidinyl group, a piperidyl group, a piperadinyl group, a morpholinyl group, a dioxolanyl group, and a dioxanyl group.

Examples of the five-membered heteroaryl group include a pyrrolyl group, a furyl group, a thienyl group, an imidazolyl group, a pyrazolyl group, an oxazolyl group, an isoxazolyl group, a triazolyl group, an isothiazolyl group, a triazolyl group, an oxadiazolyl group, a thiadiazolyl group, and a tetrazolyl group.

Examples of the six-membered heteroaryl group include a pyridyl group, a pyrazinyl group, a pyrimidinyl group, a pyridazinyl group, and a triazinyl group.

### [Het]

Het is a substituted or unsubstituted five-membered heteroaryl group.

Examples of the five-membered heteroaryl group include a pyrrolyl group, a furyl group, a thienyl group, an imidazolyl group, a pyrazolyl group, an oxazolyl group, an isoxazolyl group, a triazolyl group, an isothiazolyl group, a triazolyl group (in detail, a [1,2,3]triazolyl group, or a [1,2,4]triazolyl group), an oxadiazolyl group (in detail, a [1,2,4]oxadiazolyl group, or a [1,3,4]oxadiazolyl group), a thiadiazolyl group, and a tetrazolyl group.

Preferably, examples of the five-membered heteroaryl group include a triazolyl group, such as a 1H-1,2,4-triazol-5-yl group and a 4H-1,2,4-triazol-3-yl group; an oxadiazolyl group, such as a 1,3,4-oxadiazol-2-yl group; and a tetrazolyl group, such as a tetrazol-5-yl group.

As the substituent on the “five-membered heteroaryl group”, a halogeno group, such as a fluoro group, a chloro group, a bromo group, an iodo group; a C1-6 alkyl group, such as a methyl group, an ethyl group, a n-propyl group, an i-propyl group, a n-butyl group, a s-butyl group, an i-butyl group, a t-butyl group, a n-pentyl group, and a n-hexyl group; and a C1-6 haloalkyl group, such as a chloromethyl group, a chloroethyl group, a trifluoromethyl group, a 1,2-dichloro-n-propyl group, and a 1-fluoro-n-butyl group are preferable.

In the present invention, a 1-methyl-1H-1,2,4-triazol-5-yl group, a 5-methyl-1,3,4-oxadiazol-2-yl group, or a 1-methyl-1H-tetrazol-5-yl group is preferable as Het.

### [R¹]

R¹ is a substituted or unsubstituted C1-6 alkyl group, a substituted or unsubstituted C1-6 alkoxy group, a substituted or unsubstituted C1-6 alkylthio group, a substituted or unsubstituted C6-10 aryl group, a substituted or unsubstituted five- to six-membered heteroaryl group, or a halogeno group.

The “C1-6 alkyl group” may be a straight chain or a branched chain.

Examples of the “C1-6 alkyl group” include a methyl group, an ethyl group, a n-propyl group, a n-butyl group, a n-pentyl group, a n-hexyl group, an i-propyl group, an i-butyl group, a s-butyl group, a t-butyl group, an i-pentyl group, a neopentyl group, a 2-methylbutyl group, and an i-hexyl group.

Examples of the “C1-6 alkoxy group” include a methoxy group, an ethoxy group, a n-propoxy group, a n-butoxy group, a n-pentyloxy group, a n-hexyloxy group, an i-propoxy group, an i-butoxy group, a s-butoxy group, a t-butoxy group, and an i-hexyloxy group.

Examples of the “C1-6 alkylthio group” include a methylthio group, an ethylthio group, an n-propylthio group, a t-butylthio group, a 1-ethylpropylthio group, an n-hexylthio group, an i-hexylthio group.

Examples of the “C6-10 aryl group” include a phenyl group and a naphthyl group.

The “five- to six-membered heteroaryl group” contains one, two, three, or four heteroatoms selected from the group consisting of a nitrogen atom, an oxygen atom, and a sulfur atom as constituent atoms of the ring. When two or more heteroatoms exist, these may be the same or different.

Examples of the five-membered heteroaryl group include a pyrrolyl group, a furyl group, a thienyl group, an imidazolyl group, a pyrazolyl group, an oxazolyl group, an isoxazolyl group, a triazolyl group, an isothiazolyl group, a triazolyl group, an oxadiazolyl group, a thiadiazolyl group, and a tetrazolyl group, and examples of the six-membered heteroaryl group include a pyridyl group, a pyrazinyl group, a pyrimidinyl group, a pyridazinyl group, and a triazinyl group.

Examples of the “halogeno group” include a fluoro group, a chloro group, a bromo group, and an iodo group.

Examples of the substituent on the “C1-6 alkyl group” or the “C1-6 alkoxy group” include a halogeno group, such as a fluoro group, a chloro group, a bromo group, and an iodo group; a hydroxy group; a C1-6 alkoxy group, such as a methoxy group, an ethoxy group, a n-propoxy group, an i-propoxy group, a n-butoxy group, a s-butoxy group, an i-butoxy group, and t-butoxy group; a C1-6 haloalkoxy group, such as a 2-chloro-n-propoxy group, a 2,3-dichlorobutoxy group, and a trifluoromethoxy group; a C6-10 aryl group, such as a phenyl group and a naphthyl group; a halogeno group-, C1-6 haloalkyl group-, or C1-6 haloalkoxy group-substituted C6-10 aryl group, such as a 4-chlorophenyl group, a 4-trifluoromethylphenyl group, and a 4-trifluoromethoxyphenyl group; a cyano group.

As the substituent on the “C6-10 aryl group” or the “five- to six-membered heteroaryl group”, a halogeno group, such as a fluoro group, a chloro group, a bromo group, and an iodo group; a C1-6 alkyl group, such as a methyl group, an ethyl group, a n-propyl group, an i-propyl group, a n-butyl group, a s-butyl group, an i-butyl group, a t-butyl group, a n-pentyl group, and a n-hexyl group; a C1-6 haloalkyl group, such as a chloromethyl group, a chloroethyl group, a trifluoromethyl group, a 1,2-dichloro-n-propyl group, and a 1-fluoro-n-butyl group; a hydroxy group; a C1-6 alkoxy group, such as a methoxy group, an ethoxy group, a n-propoxy group, an i-propoxy group, a n-butoxy group, a s-butoxy group, an i-butoxy group, and a t-butoxy group; a C1-6 haloalkoxy group, such as a 2-chloro-n-propoxy group, a 2,3-dichlorobutoxy group, and a trifluoromethoxy group; a cyano group is preferable.

In the present invention, a C1-6 haloalkyl group, such as a trifluoromethyl group, is preferable as R¹.

### [R²]

R² is a hydrogen atom, a substituted or unsubstituted C1-6 alkyl group, a substituted or unsubstituted C1-6 alkylcarbonyl group, a substituted or unsubstituted C1-6 alkoxycarbonyl group, or a substituted or unsubstituted C6-10 arylcarbonyl group.

Specific examples of the “C1-6 alkyl group” include the same groups as those given as examples in R¹.

Examples of the substituent on the “C1-6 alkyl group” include a halogeno group, such as a fluoro group, a chloro group, a bromo group, and an iodo group; a hydroxy group; a C1-6 alkoxy group, such as a methoxy group, an ethoxy group, a n-propoxy group, an i-propoxy group, a n-butoxy group, a s-butoxy group, an i-butoxy group, and a t-butoxy group; a C1-6 haloalkoxy group, such as a 2-chloro-n-propoxy group, a 2,3-dichlorobutoxy group, and a trifluoromethoxy group; a C6-10 aryl group, such as a phenyl group and a naphthyl group; a halogeno group-, C1-6 haloalkyl group-, or C1-6 haloalkoxy group-substituted C6-aryl group, such as a 4-chlorophenyl group, a 4-trifluoromethylphenyl group, and a 4-trifluoromethoxyphenyl group; a C1-6 alkoxycarbonyloxy group, such as a methoxycarbonyloxy group; a cyano group.

Examples of the “C1-6 alkylcarbonyl group” include an acetyl group, a propionyl group, a butyryl group, a pentanoyl group, and a hexanoyl group.

Examples of the “C1-6 alkoxycarbonyl group” include a methoxycarbonyl group, an ethoxycarbonyl group, a n-propoxycarbonyl group, an i-propoxycarbonyl group, a n-butoxycarbonyl group, an i-butoxycarbonyl group, a s-butoxycarbonyl group, a t-butoxycarbonyl group, a n-pentyloxycarbonyl group, and a n-hexyloxycarbonyl group.

Examples of the “C6-10 arylcarbonyl group” include a benzoyl group, and a naphthylcarbonyl group.

Examples of the substituent on the “C1-6 alkylcarbonyl group” or the “C1-6 alkoxycarbonyl group” include a halogeno group, such as a fluoro group, a chloro group, a bromo group, and an iodo group; a hydroxy group; a C1-6 alkoxy group, such as a methoxy group, an ethoxy group, a n-propoxy group, an i-propoxy group, a n-butoxy group, a s-butoxy group, an i-butoxy group, and a t-butoxy group; a C1-6 haloalkoxy group, such as a 2-chloro-n-propoxy group, a 2,3-dichlorobutoxy group, and a trifluoromethoxy group; a C6-10 aryl group, such as a phenyl group and a naphthyl group;

a halogeno group-, C1-6 haloalkyl group-, or C1-6 haloalkoxy group-substituted C6-10 aryl group, such as a 4-chlorophenyl group, a 4-trifluoromethylphenyl group, and a 4-trifluoromethoxyphenyl group; a cyano group.

As the substituent on the “C6-10 arylcarbonyl group”, a halogeno group, such as a fluoro group, a chloro group, a bromo group, and an iodo group; a C1-6 alkyl group, such as a methyl group, an ethyl group, a n-propyl group, an i-propyl group, a n-butyl group, a s-butyl group, an i-butyl group, a t-butyl group, a n-pentyl group, and a n-hexyl group; a C1-6 haloalkyl group, such as a chloromethyl group, a chloroethyl group, a trifluoromethyl group, a 1,2-dichloro-n-propyl group, and a 1-fluoro-n-butyl group; a hydroxy group; a C1-6 alkoxy group, such as a methoxy group, an ethoxy group, a n-propoxy group, an i-propoxy group, a n-butoxy group, a s-butoxy group, an i-butoxy group, and a t-butoxy group; a C1-6 haloalkoxy group, such as a 2-chloro-n-propoxy group, a 2,3-dichlorobutoxy group, and a trifluoromethoxy group; a cyano group can be exemplified.

[X¹, X², R, m, and n]

X¹ is an oxygen atom or a sulfonyl group.

X² is an oxygen atom, a sulfenyl group, a sulfinyl group, a sulfonyl group, a group represented by —S(═NR)—, or a group represented by —S(═O) (═NR)—.
Here, each R independently is a hydrogen atom, a C1-6 alkyl group, a substituted or unsubstituted C1-6 alkylcarbonyl group, a substituted or unsubstituted C6-10 aryl group, or a cyano group.

m is 0 or 1, n is 0 or 1, and the sum of m and n is 1 or 2.

Specific examples of the “C1-6 alkyl group” in R include the same groups as those given as examples in R¹.

Specific examples of the “C1-6 alkylcarbonyl group” and of the substituent on the “C1-6 alkylcarbonyl group” in R include the same groups as those given as examples in R².

Specific examples of the “C6-10 aryl group” and specific examples of the substituent on the “C6-10 aryl group” in R include the same groups as those given as examples in R¹.

In the present invention, it is preferable that m is 0, and n is 1. In that case, it is preferable that X² be a sulfonyl group.

### [A]

A is a substituted or unsubstituted C2-4 alkylene group, a substituted or unsubstituted C2-3 alkenylene group, or a substituted or unsubstituted C1-2 alkyleneoxy C1-C2 alkylene group.

Examples of the “C2-4 alkylene group” include a dimethylene group, a trimethylene group, and a tetramethylene group.

Examples of the “C2-3 alkenylene group” include a vinylene group (—CH═CH—) and a propenylene group (—CH═CH—CH₂—, —CH₂—CH═CH—).

Examples of the “C1-2 alkyleneoxy C1-C2 alkylene group” include a methyleneoxymethylene group (—CH₂—O—CH₂—), a methyleneoxydimethylene group (—CH₂—O—CH₂CH₂—), a dimethyleneoxymethylene group (—CH₂CH₂—O—CH₂—), and a dimethyleneoxydimethylene group (—CH₂CH₂—O—CH₂—CH₂—).

Examples of the substituent on the “C2-4 alkylene group,” the “C2-3 alkenylene group,” or the “C1-2 alkyleneoxy C1-C2 alkylene group” include a halogeno group, such as a fluoro group, a chloro group, a bromo group, and an iodo group; a C1-6 alkyl group, such as a methyl group, an ethyl group, a n-propyl group, an i-propyl group, a n-butyl group, a s-butyl group, an i-butyl group, and a t-butyl group; a C1-6 haloalkyl group, such as a chloromethyl group, a chloroethyl group, a trifluoromethyl group, a 1,2-dichloro-n-propyl group, and a 1-fluoro-n-butyl group; a hydroxy group;

a C1-6 alkoxy group, such as a methoxy group, an ethoxy group, a n-propoxy group, an i-propoxy group, a n-butoxy group, a s-butoxy group, an i-butoxy group, and a t-butoxy group; a C1-6 haloalkoxy group, such as a 2-chloro-n-propoxy group, a 2,3-dichlorobutoxy group, and a trifluoromethoxy group; a C1-6 alkoxy C1-6 alkyl group, such as a methoxymethyl group; a C6-10 aryl group, such as a phenyl group and a naphthyl group; a halogeno group-, C1-6 haloalkyl group-, or C1-6 haloalkoxy group-substituted C6-10 aryl group, such as a 4-chlorophenyl group, a 4-trifluoromethylphenyl group, and a 4-trifluoromethoxyphenyl group; a cyano group.

### [R3, R4, Z]

R3 is a hydrogen atom, a substituted or unsubstituted C1-6 alkyl group, or a halogeno group.

R4 is a hydrogen atom, a substituted or unsubstituted C1-6 alkyl group, or a halogeno group.

Z is an oxygen atom or a sulfur atom.

Specific examples of “C1-6 alkyl group” and “halogeno group” in R³ and R⁴ include the same groups as those given as examples in R¹.

Specific examples of substituents in “C1-6 alkyl group” include the same groups as those given as examples in R¹.

In the present invention, it is preferred that R³ is a hydrogen atom, and R⁴ is a hydrogen atom.

In the present invention, it is preferred that Z is an oxygen atom.

An embodiment of the preferred compound of the present invention can be represented by the following formula (I).

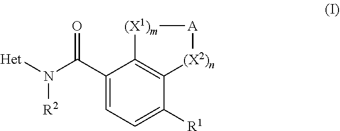

In formula (I), Het, R¹, R², A, X¹, X², m and n have the same meaning as in formula (II).

An embodiment of the preferred compound of the present invention can be represented by the following formula (III).

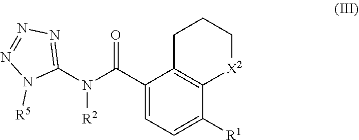

In formula (III), R¹, R² and X² have the same meaning as in formula (II).

R⁵ is a substituted or unsubstituted C1-6 alkyl group or a substituted or unsubstituted C3-6 cycloalkyl group.

Specific examples of “C1-6 alkyl group” in R⁵ include the same groups as those given as examples in R¹.

Specific examples of “C3-6 cycloalkyl group” in R⁵ include a cyclopropyl group, a cyclobutyl group, a cyclopentyl group, a cyclohexyl group.

Specific examples of substituents of “C1-6 alkyl group” and “C3-6 cycloalkyl group” include the same groups as those given as examples of the substituent of “C1-6 alkyl group” in R¹ and a C3-6 cycloalkyl group such as a cyclopropyl group.

### [Salt]

Examples of the salt of the compound (II) include a salt of an alkali metal, such as lithium, sodium, and potassium; a salt of an alkaline earth metal, such as calcium and magnesium; a salt of a transition metal, such as iron and copper; an ammonium salt; and a salt of an organic base, such as triethylamine, tributylamine, pyridine, and hydrazine.

The structure of the benzamide compound of the present invention can be determined by an NMR spectrum, an IR spectrum, an MS spectrum, and the like.

The benzamide compound of the present invention is not particularly limited by the production method therefor. In addition, the salt of the compound (II) can be obtained from the compound (II) by a known method. The benzamide compound of the present invention can be obtained by a known production method. Examples of the method for producing the benzamide compound of the present invention include methods described in Examples and the like.

The benzamide compound of the present invention exhibits a high herbicidal activity by any one of a soil treatment and a leaves-and-stems treatment in fields.

The benzamide compound of the present invention is effective for various weeds in upland fields, such as Digitaria ciliaris, Digitaria sanguinalis, Eleusine indica, Echinochloa crus-galli, Setaria viridis, Setaria glauca Setaria, Setaria faberi, Sorghum bicolor, Panicum dichotomiflorum, Urochloa platyphylla, Ipomoea hederacea, Ipomoea lacunosa, Ipomoea triloba, Calystegia japonica, Ipomoea coccinea, Cyperus rotundas, Cyperus microiria, Cyperus esculentus, Cyperus iria, Bassia scoparia, Abutilon theophrasti, Sida spinosa, Amaranthus retroflexus, Amaranthus palmeri, Amaranthus blitum, Japonica amaranthus blitum, Conyza canadensis, Conyza sumatrensis, Erigeron annuus, Xanthium occidentale, Ambrosia artemisiifolia, Ambrosia trifida, Avena sativa, Lolium multiflorum, Apera spica-venti, Bromus sterilis, Bromus japonicus, Poa annua, Alopecurus aequalis, Alopecurus myosuroides, Chenopodium album, Sagina japonica Ohwi, Matricaria chamomilla, Tripleurospermum inodorum, Galium spurium, Stellaria media, Veronica persica, Persicaria longiseta, Persicaria lapathifolia, Fallopia convolvulus, Lamium amplexicaule, and Lamium album, Lamium purpureum, and in some cases, exhibits selectivity to a crop, such as maize and wheat.

In addition, the benzamide compound of the present invention exhibits a plant growth regulation action, such as a growth inhibitory action, to a useful plant, such as a crop, an ornamental plant, and a fruit tree, in some cases.

In addition, the benzamide compound of the present invention has an excellent weed-killing effect to Echinochloa crus-galli, Cyperus difformis L., Sagittaria trifolia, Scirpus juncoides, and the like, which are paddy field weeds, and in some cases, exhibits selectivity to a rice plant.

Further, the benzamide compound of the present invention can also be applied to weed control in an orchard, a lawn, sides of a railroad, vacant land, and the like.

A herbicide of the present invention comprises as an active ingredient at least one compound or salt selected from the group consisting of the benzamide compound of the present invention, that is, the compound (II), and the salt of the compound (II).

The herbicide of the present invention may be a herbicide consisting of only the benzamide compound of the present invention, or may be formulated into a dosage form, such as, for example, a wettable powder, a granule, a powder, an emulsion, a water-soluble chemical, a suspension, or a flowable, that can generally be taken as an agricultural chemical.

A known additive or carrier can be used in formulation.

When a solid dosage form is an objective, a vegetable powder, such as soybean flour and wheat flour, a mineral fine powder, such as diatomaceous earth, apatite, gypsum, talc, bentonite, pyrophyllite, and clay, and an organic and inorganic compounds, such as sodium benzoate, urea, and salt cake, can be used.

When a liquid dosage form is an objective, a petroleum fraction, such as kerosene, xylene, and solvent naphtha, cyclohexane, cyclohexanone, dimethylformamide, dimethyl sulfoxide, an alcohol, acetone, trichloroethylene, methyl isobutyl ketone, mineral oil, vegetable oil, water, and the like can be used.

In formulation, a surfactant can be added as necessary. Examples of the surfactant include a nonionic surfactant, such as a polyoxyethylene-added alkyl phenyl ether, a polyoxyethylene-added alkyl ether, a polyoxyethylene-added higher fatty acid ester, a polyoxyethylene-added sorbitan higher fatty acid ester, and a polyoxyethylene-added tristyryl phenyl ether, a sulfuric ester salt of a polyoxyethylene-added alkyl phenyl ether, an alkylnaphthalene sulfonate, a polycarboxylate, lignosulfonate, a formaldehyde condensate of an alkylnaphthalene sulfonate, and an isobutylene-maleic anhydride copolymer.

The active ingredient concentration in the herbicide of the present invention can appropriately be set according to the dosage form. For example, the active ingredient concentration in a wettable powder is preferably 5 to 90% by weight, more preferably 10 to 85% by weight. The active ingredient concentration in an emulsion is preferably 3 to 70% by weight, more preferably 5 to 60% by weight. The active ingredient concentration in a granule is preferably 0.01 to 50% by weight, more preferably 0.05 to 40% by weight.

Soil can be treated by spraying or mixing before or after germination of weeds with a suspension or emulsion, and the granule intact, wherein a suspension or emulsion is obtained by diluting the wettable powder or emulsion obtained in this way.

When the herbicide of the present invention is applied to a farm field, a suitable amount of an active ingredient which is 0.1 g per hectare or more can be applied.

In addition, the herbicide of the present invention can also be used by mixing with known fungicide, insecticide, acaricide, herbicide, plant growth regulator, fertilizer, phytotoxicity-reducing agent (safener), or the like. Particularly when the herbicide of the present invention is used by mixing with a herbicide, the dosage can be reduced. In addition, not only such a mixed herbicide brings about labor saving, but also further higher effects expected by synergistic action. In that case, the herbicide of the present invention can be combined with a plurality of known herbicides.

An additional herbicidal active ingredient which is used in the present invention is not particularly restricted, and examples thereof include the followings.

(a) An aryloxy phenoxy propionic acid ester type, such as clodinafop-propargyl, cyhalofop-butyl, diclofop-methyl, fenoxaprop-P-ethyl, fluazifop-P, fluazifop-P-butyl, haloxyfop-methyl, pyriphenop-sodium, propaquizafop, quizalofop-P-ethyl, and metamifop; a cyclohexanedione type, such as alloxydim, butroxydim, clethodim, cycloxydim, profoxydim, sethoxydim, tepraloxydim, and tralkoxydim; a phenypyrazoline type, such as pinoxaden; and other herbicidal active ingredients which are each considered to exhibit herbicidal effectiveness by inhibiting acetyl CoA carboxylase of a plant.

(b) A sulfonyl urea type, such as amidosulfuron, azimsulfuron, bensulfuron-methyl, chlorimuron-ethyl, chlorsulfuron, cinosulfuron, cyclosulfamuron, ethametsulfuron-methyl, ethoxysulfuron, flazasulfuron, flupyrsulfuron, foramsulfuron, halosulfuron-methyl, imazosulfuron, iodosulfuron-methyl, mesosulfuron, mesosulfuron-methyl, metsulfuron-methyl, nicosulfuron, oxasulfuron, primisulfuron, prosulfuron, pyrazosulfuron-ethyl, rimsulfuron, sulfometuron-methyl, sulfosulfuron, thifensulfuron-methyl, triasulfuron, tribenuron-methyl, trifloxysulfuron, triflusulfuron-methyl, tritosulfuron, orthosulfamuron, propyrisulfuron, flucetosulfuron, metazosulfuron, methiopyrsulfuron, monosulfuron-methyl, orsosulfuron, and iofensulfuron; an imidazolinone type, such as imazapic, imazamethabenz, imazamox-ammonium, imazapyr, imazaquin, and imazethapyr; a triazolopyrimidine sulfonamide type, such as cloransulam-methyl, diclosulam, florasulam, flumetsulam, metosulam, penoxsulam, pyroxsulam, and metosulfam; a pyrimidinyl(thio)benzoate type, such as bispyribac-sodium, pyribenzoxim, pyriftalid, pyrithiobac-sodium, pyriminobac-methyl, and pyrimisulfan; a sulfonylaminocarbonyltriazolinone type, such as flucarbazone, propoxycarbazone, and thiencarbazone-methyl; a sulfonanilide type, such as triafamone; and other herbicidal active ingredients which are each considered to exhibit herbicidal effectiveness by inhibiting acetolactate synthase (ALS) (acetohydroxy acid synthase (AHAS)) of a plant.

(c) A triazine type, such as ametryn, atrazine, cyanazine, desmetryne, dimethametryn, prometon, prometryn, a propazine type (propazine), CAT (simazine), simetryn, terbumeton, terbuthylazine, terbutryne, trietazine, atratone, and cybutryne; a triazinone type, such as hexazinone, metamitron, and metribuzin; a triazolinone type, such as amicarbazone; a uracil type, such as bromacil, lenacil, and terbacil; a pyridazinone type, such as PAC (chloridazon); a carbamate type, such as desmedipham, phenmedipham, and swep; a urea type, such as chlorobromuron, chlorotoluron, chloroxuron, dimefuron, DCMU (diuron), ethidimuron, fenuron, fluometuron, isoproturon, isouron, linuron, methabenzthiazuron, metobromuron, metoxuron, monolinuron, neburon, siduron, tebuthiuron, metobenzuron, and karbutilate; an amide type, such as DCPA (propanil), and CMMP (pentanochlor); an anilide type, such as cypromid; a nitrile type, such as bromofenoxim, bromoxynil, and ioxynil; a benzothiadiazinone type, such as bentazone; a phenylpyridazine type, such as pyridate and pyridafol; and other herbicidally active ingredients, such as methazole, which are each considered to exhibit herbicidal effectiveness by inhibiting photosynthesis of a plant.

(d) A bipyridylium type, such as diquat and paraquat; and other herbicidal active ingredients which are each considered to exhibit prompt herbicidal effectiveness in such a way that the herbicidal active ingredient itself converts into a free radical in a plant body to produce active oxygen.

(e) A diphenyl ether type, such as acifluorfen-sodium, bifenox, chlomethoxynil (chlomethoxyfen), fluoroglycofen, fomesafen, halosafen, lactofen, oxyfluorfen, nitrofen, and ethoxyfen-ethyl; a phenylpyrazole type, such as fluazolate and pyraflufen-ethyl; an N-phenylphthalimide type, such as cinidon-ethyl, flumioxazin, flumiclorac-pentyl, and chlorphthalim; a thiadiazole type, such as fluthiacet-methyl and thidiazimin; an oxadiazole type, such as oxadiazon and oxadiargyl; a triazolinone type, such as azafenidin, carfentrazone-ethyl, sulfentrazone, and bencarbazone; an oxazolidinedione type, such as pentoxazone; a pyrimidinedione type, such as benzfendizone and butafenacil; a sulfonylamide type, such as saflufenacil; a pyridazine type, such as flufenpyr-ethyl; and other herbicidally active ingredients, such as pyrachlonil, profluazol, tiafenacil, and trifludimoxazin, which are each considered to exhibit herbicidal effectiveness by inhibiting chlorophyll biosynthesis of a plant to accumulate a photosensitizing peroxide substance abnormally in a plant body.

(f) A pyridazinone type, such as norflurazon and metflurazon; a pyridinecarboxamide type, such as diflufenican and picolinafen; a triketone type, such as mesotrione, sulcotrione, tefuryltrione, tembotrione, bicyclopyrone, and fenquinotrione; an isoxazole type, such as isoxachlortole and isoxaflutole; a pyrazole type, such as benzofenap, pyrazolate (pyrazolynate), pyrazoxyfen, topramezone, pyrasulfotole, and tolpyralate; a triazole type, such as ATA (amitrol); an isoxazolidinone type, such as clomazone; a diphenyl ether type, such as aclonifen; and other herbicidally active ingredients, such as beflubutamid, fluridone, flurochloridone, flurtamone, benzobicyclone, methoxyphenone, and ketospiradox, which are each considered to exhibit herbicidal effectiveness, which is characterized by a whitening action, by inhibiting plant pigments, such as carotenoids.

(g) A glycine type, such as glyphosate, glyphosate-ammonium, glyphosate-isopropylamine, and glyphosate trimesium (sulfosate); and other EPSP synthase inhibitors.

(h) Glutamine synthase inhibitors of a phosphinic acid type and the like, such as glufosinate, glufosinate-ammonium, and bialaphos (bilanafos), and other herbicidal active ingredients which are each considered to exhibit herbicidal effectiveness by inhibiting amino acid biosynthesis of a plant.

(i) A carbamate type, such as asulam; and other DHP (dihydropteroic acid) synthase inhibitors.

(j) A dinitroaniline type, such as bethrodine (benfluralin), butralin, dinitramine, ethalfluralin, oryzalin, pendimethalin, trifluralin, nitralin, and prodiamine; a phosphoramidate type, such as amiprofos-methyl and butamifos; a pyridine type, such as dithiopyr and thiazopyr; a benzamide type, such as propyzamide and tebutam; a benzoic acid type, such as chlorthal and TCTP (chlorthal-dimethyl); a carbamate type, such as IPC (chlorpropham), propham, carbetamide, and barban; an arylalanine type, such as flamprop-M and flamprop-M-isopropyl; a chloroacetamide type, such as acetochlor, alachlor, butachlor, dimethachlor, dimethenamid, dimethenamid-P, metazachlor, metolachlor, S-metolachlor, pethoxamid, pretilachlor, propachlor, propisochlor, and thenylchlor; an acetamide type, such as diphenamid, napropamide, and naproanilide; an oxyacetamide type, such as flufenacet and mefenacet; a tetrazolinone type, such as fentrazamide; and other herbicidally active ingredients, such as anilofos, indanofan, cafenstrole, piperophos, methiozolin, fenoxasulfone, pyroxasulfone, and ipfencarbazone, which are each considered to exhibit herbicidal effectiveness by inhibiting microtubule polymerization, microtubule formation, or cell division of a plant, or by inhibiting Very Long Chain Fatty Acid (VLCFA) biosynthesis of a plant.

(k) A nitrile type, such as DBN (dichlobenil) and DCBN (chlorthiamid); a benzamide type, such as isoxaben; a triazolocarboxamide type, such as flupoxam; a quinolinecarboxylic acid type, such as quinclorac; and other herbicidally active ingredients, such as triaziflam and indaziflam, which are each considered to exhibit herbicidal effectiveness by inhibiting cell wall (cellulose) synthesis.

(l) A dinitrophenol type, such as DNOC, DNBP (dinoseb), and dinoterb; and other herbicidally active ingredients which are each considered to exhibit herbicidal effectiveness by uncoupling (membrane disruption).

(m) A thiocarbamate type, such as butylate, hexylthiocarbam (cycloate), dimepiperate, EPIC, esprocarb, molinate, orbencarb, pebulate, prosulfocarb, benthiocarb (thiobencarb), tiocarbazil, triallate, vernolate, and diallate; a phosphorodithioate type, such as SAP (bensulide); a benzofuran type, such as benfuresate and ethofumesate; a chlorocarbonic acid type, such as TCA, DPA (dalapon), and tetrapion (flupropanate); and other herbicidal active ingredients which are each considered to exhibit herbicidal effectiveness by inhibiting lipid biosynthesis of a plant.

(n) A phenoxycarboxylic acid type, such as clomeprop, 2,4-PA (2,4-D), 2,4-DB, dichlorprop, MCPA, MCPB, and MCPP (mecoprop); a benzoic acid type, such as chloramben, MDBA (dicamba), and TCBA (2,3,6-TBA); a pyridinecarboxylic acid type, such as clopyralid, aminopyralid, fluroxypyr, picloram, triclopyr and halauxifen; a quinolinecarboxylic acid type, such as quinclorac and quinmerac; a phthalamate semicarbazone type, such as NPA (naptalam) and diflufenzopyr; and other herbicidally active ingredients, such as benazolin, diflufenzopyr, fluroxypyr, chlorflurenol, aminocyclopyrachlor, and DAS534, which are each considered to exhibit herbicidal effectiveness by disturbing a hormone action of a plant.

(o) An arylaminopropionic acid type, such as flamprop-M-methyl/flamprop-isopropyl; a pyrazolium type, such as difenzoquat; an organoarsenic type, such as DSMA and MSMA; and other herbicides, such as bromobutide, chlorflurenol, cinmethylin, cumyluron, dazomet, daimuron, methyl-dymron, etobenzanid, fosamine, oxaziclomefone, oleic acid, pelargonicacid, pyributicarb, endothall, sodiumchlorate, metam, quinoclamine, cyclopyrimorate, tridiphane, and clacyfos.

Examples of the safener which can be used in the present invention include benoxacor, cloquintocet, cloquintocet-mexyl, cyometrinil, cyprosulfamide, dichlormid, dicyclonon, dietholate, fenchlorazole, fenchlorazole-ethyl, fenclorim, flurazole, fluxofenim, furilazole, isoxadifen, isoxadifen-ethyl, mefenpyr, mefenpyr-diethyl, mephenate, naphthalic anhydride, and oxabetrinil.

### FORMULATION EXAMPLES

Some Formulation Examples relating to the herbicide of the present invention will be given, but active ingredient compounds, additives, and addition ratios are not limited to only the present Examples and can be modified in a wide range. Part or parts in the Formulation Examples is part or parts by weight.

### (Formulation Example 1) Wettable Powder

| | |
|---|---|
| Compound of the present invention | 20 parts |
| White carbon | 20 parts |
| Diatomaceous earth | 52 parts |
| Sodium alkylsulfate | 8 parts |

The above substances are uniformly mixed and finely pulverized to obtain a wettable powder containing 20% of an active ingredient.

### (Formulation Example 2) Emulsion

| | |
|---|---|
| Compound of the present invention | 20 parts |
| Xylene | 55 parts |
| Dimethylformamide | 15 parts |
| Polyoxyethylene phenyl ether | 10 parts |

The above substances are mixed and dissolved to obtain an emulsion containing 20% of an active ingredient.

### (Formulation Example 3) Granule

| | |
|---|---|
| Compound of the present invention | 5 parts |
| Talc | 40 parts |
| Clay | 38 parts |
| Bentonite | 10 parts |
| Sodium alkylsulfate | 7 parts |

The above substances are uniformly mixed and finely pulverized, and a resultant mixture is thereafter granulated into a granule having a diameter of 0.5 to 1.0 mm to obtain a granule containing 5% of an active ingredient.

Next, Synthesis Examples will be described. However, the present invention is not limited to the Examples described below.

### Example 1

### Synthesis of N-(1-Methyl-1H-tetrazol-5-yl)-8-(trifluoromethyl)thiochromane-5-carboxamide (Compound Number a-37)

### (Step 1)

### Synthesis of 3-(6-Bromo-2-fluoro-3-(trifluoromethyl)phenyl)propanoic acid

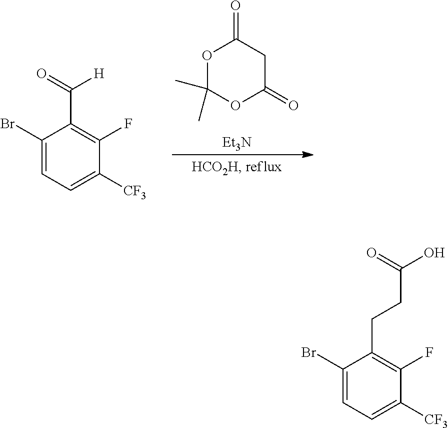

To a 500 mL four-necked flask, formic acid (58.2 g), triethylamine (18.3 g), 6-bromo-2-fluoro-3-(trifluoromethyl)benzaldehyde (48.7 g) and Meldrum's acid (26.0 g) were added in sequence at 0° C., and a resultant mixture was thereafter heated to reflux for four hours.

After hydrochloric acid was added to a resultant reaction solution, a precipitated crystal was separated by filtration, and a resultant crystal was dried, thereby obtaining 53.8 g of a title compound.

### (Step 2)

### Synthesis of 3-(6-Bromo-2-fluoro-3-(trifluoromethyl)phenyl)propan-1-ol

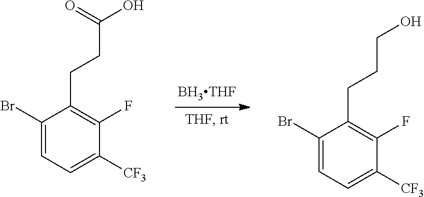 [SMILES: O=C(O)CCc1c(Br)ccc(C(F)(F)F)c1F ; OCCCc1c(Br)ccc(C(F)(F)F)c1F ; "BH3·THF"]

In tetrahydrofuran (191 mL), 3-(6-bromo-2-fluoro-3-(trifluoromethyl)phenyl)propionic acid (30 g) was dissolved, and a resultant solution was stirred at room temperature. A borane-tetrahydrofuran complex (0.9 M, 127 mL) was added thereto, and a resultant solution was stirred at 0° C. for one hour.

A resultant reaction solution was poured into hydrochloric acid, and extraction was then performed with ethyl acetate. A resultant organic layer was washed with saturated brine, dried over anhydrous magnesium sulfate, and filtrated. A filtrate was concentrated in vacuo, and a resultant residue was purified by silica gel chromatography to obtain 28.9 g of a title compound.

### (Step 3)

### Synthesis of 1-Bromo-2-(3-chloropropyl)-3-fluoro-4-(trifluoromethyl)benzene

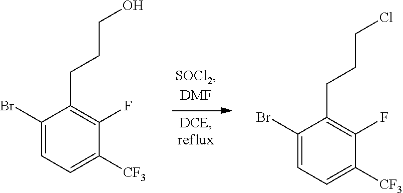 [SMILES: OCCCc1c(Br)ccc(C(F)(F)F)c1F ; Fc1c(C(F)(F)F)ccc(Br)c1CCCCl]

In dichloroethane (102 mL), 3-(6-bromo-2-fluoro-3-(trifluoromethyl)phenyl)propan-1-ol (15.3 g) was dissolved, and a resultant solution was stirred at room temperature. Thionyl chloride (9.1 g) and N,N-dimethylformamide (0.2 g) were added thereto, and a resultant solution was heated to reflux for two hours.

A resultant reaction solution was concentrated in vacuo, and a resultant residue was purified by silica gel chromatography to obtain 16.4 g of a title compound.

### (Step 4)

### Synthesis of 5-Bromo-8-(trifluoromethyl)thiochromane

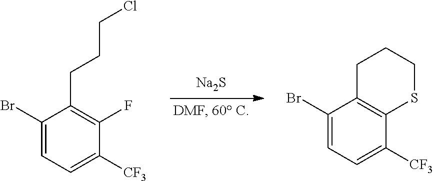 [SMILES: Fc1c(C(F)(F)F)ccc(Br)c1CCCCl ; "Na2S" ; FC(F)(F)c1ccc(Br)c2c1SCCC2]

In N,N-dimethylformamide (158 mL), 1-bromo-2-(3-chloropropyl)-3-fluoro-4-(trifluoromethyl)benzene (14.4 g) was dissolved, and a resultant solution was stirred at room temperature. Sodium sulfide (4.2 g) was added thereto, and a resultant solution was heated to reflux at 60° C. overnight.

A resultant reaction solution was poured into water, and then extracted with ethyl acetate. A resultant organic layer was washed with saturated brine, dried over anhydrous magnesium sulfate, and filtrated. A filtrate was concentrated in vacuo, and a resultant residue was purified by silica gel chromatography to obtain 6.8 g of a title compound.

### (Step 5)

### Synthesis of 8-(Trifluoromethyl)thiochromane-5-carboxylic acid

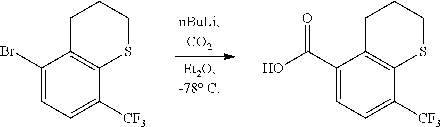 [SMILES: FC(F)(F)c1ccc(Br)c2c1SCCC2 ; O=C(O)c1ccc(C(F)(F)F)c2c1CCCS2]

In diethyl ether (56 mL), 5-bromo-8-(trifluoromethyl)thiochromane (5.0 g) was dissolved, and a resultant solution was stirred at −78° C. n-Butyl lithium (2.76 M, 6.1 mL) was added thereto, and a resultant solution was stirred further for 30 minutes. Thereafter, dry ice (4.0 g) was added at the same temperature to elevate the temperature gradually to room temperature.

A resultant reaction solution was poured into hydrochloric acid, and then extracted with ethyl acetate. A resultant organic layer was washed with saturated brine, dried over anhydrous magnesium sulfate, and filtrated. A filtrate was concentrated in vacuo to obtain a title compound (3.5 g).

### (Step 6)

### Synthesis of N-(1-Methyl-1H-tetrazol-5-yl)-8-(trifluoromethyl) thiochromane-5-carboxamide

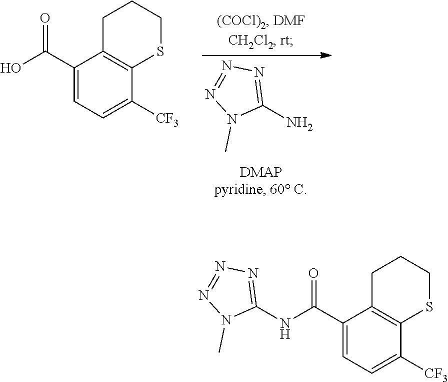

In dichloroethane (10 mL), 8-(trifluoromethyl)thiochromane-5-carboxylic acid (0.73 g) was dissolved, and a resultant solution was stirred at room temperature. Oxalyl chloride (0.48 g) and one drop of N,N-dimethylformamide were added thereto, and a resultant solution was stirred at room temperature for one hour. A resultant reaction solution was concentrated in vacuo. The residue was used for the next reaction without purification.

A resultant residue was dissolved in pyridine, and a resultant solution was stirred at room temperature. 1-Methyl-1H-tetrazol-5-amine (0.3 g) and N,N-dimethyl-4-aminopyridine (30 mg) were added thereto, and a resultant solution was stirred at 60° C. overnight.

A resultant reaction solution was concentrated in vacuo, and a resultant residue was purified by silica gel chromatography to obtain a title compound (0.48 g).

Examples of the compound of the present invention, produced by the same method as in the Synthetic Example, are shown in Tables 1 and 2. Table 1 shows substituents in the compound of formula (I). Table 1 also shows melting points together with the substituents.

In the column of Het in Table 1, “Het-1” is a 1-methyl-1H-tetrazol-5-yl group, “Het-2” is a 5-methyl-1,3,4-oxadiazol-2-yl group, and “Het-3” is a 1-methyl-1H-1,2,4-triazol-5-yl group. In Table 1, “Ac” is an acetyl group, “Bn” is a benzyl group, “Ph” is a phenyl group, “Me” is a methyl group, “Bz” is a benzoyl group, and “Et” is an ethyl group.

TABLE 1

| Compound | | | | (X¹) | | | Melting |
|---|---|---|---|---|---|---|---|
| number | Het | R² | R¹ | m | A | (X²)n | point |
| a-1 | Het-1 | H | CF₃ | — | CH₂CH₂ | S(═O) | * |
| a-2 | Het-2 | H | CF₃ | — | CH₂H₂ | S | 217-219° C. |
| a-3 | Het-1 | H | CF₃ | — | CH₂H₂ | S | * |
| a-4 | Het-1 | Ac | CF₃ | — | CH₂H₂ | SO₂ | * |
| a-5 | Het-1 | Bn | CF₃ | — | CH₂H₂ | SO₂ | * |
| a-6 | Het-1 | H | CF₃ | — | CH₂H₂ | SO₂ | 273-275° C. |
| a-7 | Het-1 | H | SMe | — | CH₂H₂ | SO₂ | 242-243° C. |
| a-8 | Het-2 | H | OMe | — | CH₂H₂ | SO₂ | 253-254° C. |
| a-9 | Het-1 | H | OMe | — | CH₂H₂ | SO₂ | 271-272° C. |
| a-10 | Het-3 | H | CF₃ | — | CH₂H₂ | SO₂ | 190-191° C. |
| a-11 | Het-1 | H | CF₃ | — | CH₂H₂ | SO₂ | 253-254° C. |
| a-12 | Het-2 | H | CF₃ | — | CH₂H₂ | SO₂ | 237-238° C. |
| a-13 | Het-2 | H | CF₃ | SO₂ | CH₂H₂ | — | 161-163° C. |
| a-14 | Het-1 | H | CF₃ | — | CH═CH | S | 208-209° C. |
| a-15 | Het-2 | H | CF₃ | — | CH═CH | S | 216-219° C. |
| a-16 | Het-1 | H | CF₂H | — | CH₂H₂H₂ | SO₂ | 253-255° C. |
| a-17 | Het-1 | H | Et | — | CH₂H₂H₂ | SO₂ | 246-247° C. |
| a-18 | Het-1 | H | Et | — | CH₂H₂H₂ | S | 170-173° C. |
| a-19 | Het-1 | H | (4-MeOPh) | — | CH₂H₂H₂ | SO₂ | 278-280° C. |
| a-20 | Het-1 | H | (4-MeOPh) | — | CH₂H₂H₂ | S | 216-218° C. |
| a-21 | Het-1 | H | CF₃ | — | CH₂H₂H₂ | S(═O)(═N—CN) | 93-94° C. |
| a-22 | Het-1 | H | CF₃ | — | CH₂H₂H₂ | S(═N—CN) | 182-184° C. |
| a-23 | Het-1 | H | Br | — | CH₂H₂H₂ | SO₂ | 240-243° C. |
| a-24 | Het-1 | H | Br | — | CH₂H₂H₂ | S | 200-203° C. |
| a-25 | Het-1 | CH₂(4-MeOPh) | CF₃ | — | CH₂H₂H₂ | S(═O)(═NH) | * |
| a-26 | Het-1 | Bz | CF₃ | — | CH₂H₂H₂ | SO₂ | * |
| a-27 | Het-1 | CH(Me)OCO₂Me | CF₃ | — | CH₂H₂H₂ | SO₂ | * |
| a-28 | Het-1 | H | CF₃ | — | CH₂H₂H₂ | S(═O)(═NH) | 122-124° C. |
| a-29 | Het-1 | CH₂OMe | CF₃ | — | CH₂H₂H₂ | SO₂ | * |
| a-30 | Het-1 | Bn | CF₃ | — | CH₂H₂H₂ | SO₂ | 180-181° C. |
| a-31 | Het-3 | H | CF₃ | — | CH₂H₂H₂ | | 150-151° C. |
| a-32 | Het-2 | H | CF₃ | — | CH₂H₂H₂ | S | 203-205° C. |
| a-33 | Het-3 | H | CF₃ | — | CH₂H₂H₂ | SO₂ | 231-233° C. |
| a-34 | Het-2 | H | CF₃ | — | CH₂H₂H₂ | SO₂ | 247-249° C. |
| a-35 | Het-1 | H | CF₃ | — | CH₂H₂H₂ | SO₂ | * |
| a-36 | Het-1 | H | CF₃ | — | CH₂H₂H₂ | S(═O) | * |
| a-37 | Het-1 | H | CF₃ | — | CH₂H₂H₂ | S | 166-167° C. |
| a-38 | Het-1 | H | CF₃ | — | CH₂H₂H₂ | SO₂ | 193-194° C. |
| a-39 | Het-1 | H | CF₃ | O | CH₂H₂ | SO₂ | 221-223° C. |
| a-40 | Het-1 | H | CF₃ | O | CH₂H₂ | S | 130-131° C. |
| a-41 | Het-2 | H | CF₃ | — | CH₂H₂H₂ | O | 224-226° C. |
| a-42 | Het-1 | H | CF₃ | — | CH₂H₂H₂ | O | 91-93° C. |
| a-43 | Het-1 | H | CF₃ | — | CH₂H₂H₂H₂ | SO₂ | 194-195° C. |
| a-44 | Het-1 | H | CF₃ | — | CH₂H₂H₂H₂ | S(═O) | 115-116° C. |
| a-45 | Het-2 | H | CF₃ | — | CH₂H₂H₂H₂ | SO₂ | 102-103° C. |
| a-46 | Het-2 | H | CF₃ | — | CH₂H₂H₂H₂ | S | 220-221° C. |
| a-47 | Het-1 | H | CF₃ | — | CH₂H₂H₂H₂ | S | 173-174° C. |

TABLE 2

| Com- | | |
|---|---|---|
| pound | | |
| number | Structural formula | Melting point |
| b-1 | 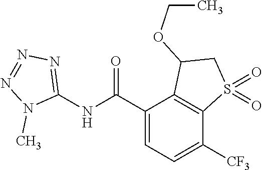 [SMILES: CCOC1CS(=O)(=O)c2c(C(F)(F)F)ccc(C(=O)Nc3nnnn3C)c21] | 209-211° C. |
| b-2 | 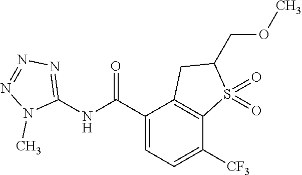 [SMILES: COCC1Cc2c(C(=O)Nc3nnnn3C)ccc(C(F)(F)F)c2S1(=O)=O] | 215-217° C. |
| b-3 | 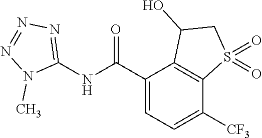 [SMILES: Cn1nnnc1NC(=O)c1ccc(C(F)(F)F)c2c1C(O)CS2(=O)=O] | 202-205° C. |
| b-4 | 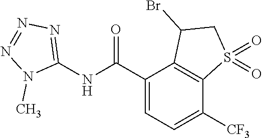 [SMILES: Cn1nnnc1NC(=O)c1ccc(C(F)(F)F)c2c1C(Br)CS2(=O)=O] | 209-211° C. |
| b-5 | 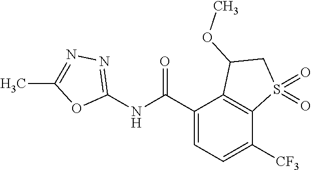 [SMILES: COC1CS(=O)(=O)c2c(C(F)(F)F)ccc(C(=O)Nc3nnc(C)o3)c21] | 206-207° C. |
| b-6 | 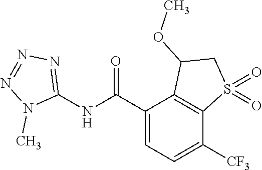 [SMILES: COC1CS(=O)(=O)c2c(C(F)(F)F)ccc(C(=O)Nc3nnnn3C)c21] | * |
| b-7 | 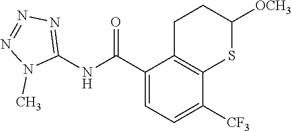 [SMILES: COC1CCc2c(C(=O)Nc3nnnn3C)ccc(C(F)(F)F)c2S1] | * |
| b-8 | 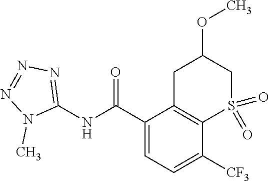 [SMILES: COC1Cc2c(C(=O)Nc3nnnn3C)ccc(C(F)(F)F)c2S(=O)(=O)C1] | 226-228° C. |
| b-9 | 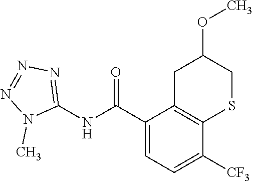 [SMILES: COC1CSc2c(C(F)(F)F)ccc(C(=O)Nc3nnnn3C)c2C1] | 168-170° C. |
| b-10 | 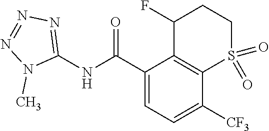 [SMILES: Cn1nnnc1NC(=O)c1ccc(C(F)(F)F)c2c1C(F)CCS2(=O)=O] | 220-223° C. |
| b-11 | 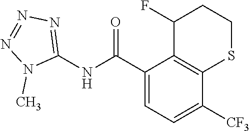 [SMILES: Cn1nnnc1NC(=O)c1ccc(C(F)(F)F)c2c1C(F)CCS2] | 132-135° C. |
| b-12 | 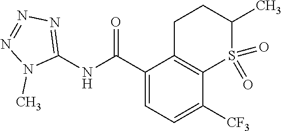 [SMILES: CC1CCc2c(C(=O)Nc3nnnn3C)ccc(C(F)(F)F)c2S1(=O)=O] | 270-271° C. |
| b-13 | 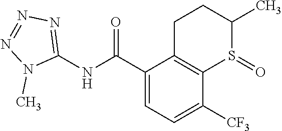 [SMILES: CC1CCc2c(C(=O)Nc3nnnn3C)ccc(C(F)(F)F)c2S1=O] | * |
| b-14 | 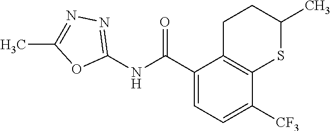 | 182-183° C. |
| b-15 | 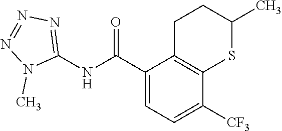 [SMILES: CC1CCc2c(C(=O)Nc3nnnn3C)ccc(C(F)(F)F)c2S1] | * |
| b-16 | 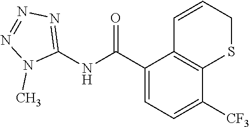 [SMILES: Cn1nnnc1NC(=O)c1ccc(C(F)(F)F)c2c1C=CCS2] | * |
| b-17 | 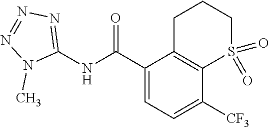 [SMILES: Cn1nnnc1NC(=O)c1ccc(C(F)(F)F)c2c1CCCS2(=O)=O] | * |
| b-18 | 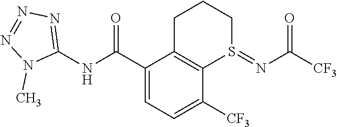 [SMILES: Cn1nnnc1NC(=O)c1ccc(C(F)(F)F)c2c1CCC/S2=N\C(=O)C(F)(F)F] | * |
| b-19 | 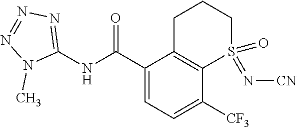 [SMILES: Cn1nnnc1NC(=O)c1ccc(C(F)(F)F)c2c1CCCS2(=O)=NC#N] | 151-153° C. |
| b-20 | 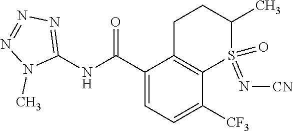 [SMILES: CC1CCc2c(C(=O)Nc3nnnn3C)ccc(C(F)(F)F)c2S1(=O)=NC#N] | * |
| b-21 | 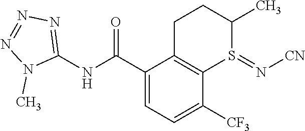 [SMILES: CC1CCc2c(C(=O)Nc3nnnn3C)ccc(C(F)(F)F)c2/S1=N/C#N] | 117-120° C. |
| b-22 | 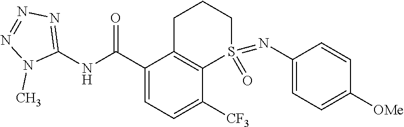 [SMILES: COc1ccc(N=S2(=O)CCCc3c(C(=O)Nc4nnnn4C)ccc(C(F)(F)F)c32)cc1] | 123-124° C. |
| b-23 | 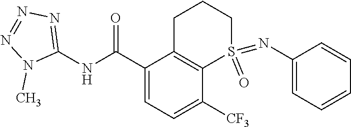 [SMILES: Cn1nnnc1NC(=O)c1ccc(C(F)(F)F)c2c1CCCS2(=O)=Nc1ccccc1] | 110-112° C. |
| b-24 | 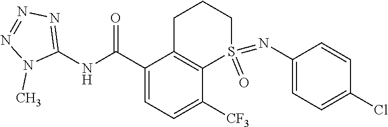 [SMILES: Cn1nnnc1NC(=O)c1ccc(C(F)(F)F)c2c1CCCS2(=O)=Nc1ccc(Cl)cc1] | 113-114° C. |

Among the compounds described in Tables 1 and 2, the compounds of the compound numbers of a-6 and a-35 are sodium salts.

Among the compounds described in Tables 1 and 2, the compounds with * marked in the column of melting point were compounds having an amorphous or viscous oil characteristic. The ¹H-NMR data thereof are shown in Table 3.

TABLE 3

| Compound | |
|---|---|
| number | 1H-NMR Data |
| a-1 | 1H-NMR(400 MHz, CDCl3): δ 3.10-3.17(m, 1H), 3.75-4.05(m, 5H), 4.19(dd, |
| | 1H), 7.70(d, 1H), 8.13(d, 1H), 11.91(bs, 1H). |
| a-3 | 1H-NMR(400 MHz, CDCl3): δ 3.40-3.45(m, 2H), 3.67(t, 2H), 4.09(s, |
| | 3H), 7.65(d, 1H), 7.83(d, 1H), 11.32(bs, 1H). |
| a-4 | 1H-NMR(400 MHz, CDCl3): δ 2.36(s, 3H), 3.55-3.59(m, 2H), 3.75-3.82(m, |
| | 2H), 4.03(s, 3H), 7.97(d, 1H), 8.49(d, 1H). |
| a-5 | 1H-NMR(400 MHz, CDCl3): δ 3.46(t, 2H), 3.68(t, 2H), 3.88(s, 3H), 5.53(s, |
| | 2H), 7.25-7.32(m, 5H), 7.73(d, 1H), 8.29(d, 1H). |
| a-25 | 1H-NMR(400 MHz, CDCl3): δ 2.25-2.60(m, 4H), 2.87(s, 3H), 2.94(s, 3H), |
| | 3.20-3.35(m, 2H), 3.80(s, 2H), 4.61(d, 1H), 4.83-4.88(m, 1H), 6.74-6.89(m, |
| | 4H), 7.58(d, 1H), 7.90(d, 1H), 8.00(s, 1H). |
| a-26 | 1H-NMR(400 MHz, CDCl3): δ 2.58(td, 2H), 3.34(t, 2H), 3.43-3.49(m, 2H), |
| | 4.01(s, 3H), 7.44(t, 2H), 7.59-7.65(m, 3H), 7.78(dd, 2H). |
| a-27 | 1H-NMR(400 MHz, CDCl3): δ 2.01(d, 3H), 2.47-2.53(m, 2H), 3.37-3.46(m, |
| | 4H), 3.79(s, 3H), 3.92(s, 3H), 7.06(q, 1H), 7.81(d, 1H), 8.02(d, 1H). |
| a-29 | 1H-NMR(400 MHz, CDCl3): δ 2.46-2.53(m, 2H), 3.38-3.43(m, 7H), 3.94(s, |
| | 3H), 5.76(s, 2H), 7.79(d, 1H), 7.92(d, 1H). |
| a-35 | 1H-NMR(400 MHz, DMSOd-6): δ 2.26-2.32(m, 2H), 3.30-3.41(m, |
| | 2H), 3.53-3.57(m, 2H), 3.68(s, 3H), 7.70(d, 1H), 7.79(d, 1H). |
| a-36 | 1H-NMR(400 MHz, CDCl3): δ 2.03-2.10(m, 1H), 2.56-2.72(m, 2H), 3.16- |
| | 3.35(m, 2H), 3.42-3.46(m, 1H), 4.08(s, 3H), 7.68(d, 1H), 7.80(d, 1H), |
| | 11.26(bs, 1H). |
| b-6 | 1H-NMR(400 MHz, CDCl3): δ 3.48(s, 3H)3.77-3.79(m, 2H), 4.13(s, |
| | 3H), 5.66(dd, 1H), 8.04(d, 1H), 8.31(d, 1H). |
| b-7 | 1H-NMR(400 MHz, CDCl3): δ 2.08-2.15(m, 1H), 2.25-2.35(m, 1H), 2.82- |
| | 2.90(m, 1H), 3.22-3.30(m, 1H), 3.43(s, 3H), 4.11(s, 3H), 5.03-5.06(m, 1H), |
| | 7.57(d, 1H), 7.68(d, 1H), 10.20(s, 1H). |
| b-13 | 1H-NMR(400 MHz, DMSO-d6): δ 0.96(d, 3H), 1.70-1.78(m, 1H), 2.63- |
| | 2.72(m, 1H), 3.03-3.11(m, 1H), 3.21-3.26(m, 1H), 3.48-3.54(m, 1H), |
| | 3.99(s, 3H), 7.93(d, 1H), 7.99(d, 1H), 11.86(s, 1H). |
| b-15 | 1H-NMR(400 MHz, CDCl3): δ 1.39(d, 3H), 1.65-1.75(m, 1H), 2.17-2.23(m, |
| | 1H), 2.97-3.14(m, 2H), 3.37-3.42(m, 1H), 4.10(s, 3H), 7.50(d, 1H), 7.63(d, |
| | 1H), 10.82(s, 1H). |
| b-16 | 1H-NMR(400 MHz,CDCl3): δ 3.38(d, 2H), 4.11(s, 3H), 6.22-6.27(m, 1H), |
| | 6.89(d, 1H), 7.69(dd, 2H), 11.05(bs, 1H). |
| b-17 | 1H NMR (400 MHz, DMSO-d6): δ2.35 (m, 2H), 3.22 (m, 2H), 3.62 (m, 2H), |
| | 3.96 (s, 3H), 7.74 (d, 1H), 7.92 (d, 1H). |
| b-18 | 1H NMR (400 MHz, MeOH-d4): δ2.40-2.60 (m, 2H), 3.30-3.50 (m, 2H), |
| | 3.80-3.90 (m, 2H), 4.07 (s, 3H), 7.90-8.12 (m, 2H). |
| b-20 | 1H NMR (400 MHz, MeOH-d4): δ1.56-1.62 (m, 3H), 2.37-2.50 (m, 2H), |
| | 2.76 (m, 1H), 3.30-3.50 (m, 2H), 4.07 (s, 3H), 8.13 (bs, 2H). |

Examples of the compound of the present invention, produced by the same method as in the Synthetic Example, are shown in Table 4. In Table 4, the substituents in the compound of formula (III) are shown together with the melting points.

In Table 4, “^{c}Pr” is a cyclopropyl group, “ⁱPr” is an isopropyl group, “Et” is an ethyl group, “^{t}Bu” is a t-butyl group, “Me” is a methyl group, “ⁿPr” is an n-propyl group, “Bz” is a benzoyl group, “Ac” is an acetyl group, and “Ph” is a phenyl group.

TABLE 4

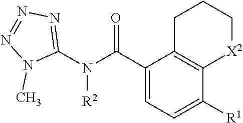

| Com- | | | | | |
|---|---|---|---|---|---|
| pound | | | | | |
| number | R¹ | R² | R⁵ | X² | Melting point |
| c-1 | CF₃ | H | CHF₂CH₂ | SO₂ | 119-121° C. |
| c-2 | CF₃ | H | CHF₂CH₂ | S | 149-152° C. |
| c-3 | CF₃ | H | ^{c}PrCH₂ | SO₂ | 144-148° C. |
| c-4 | CF₃ | H | ^{c}PrCH₂ | S | 175-177° C. |
| c-5 | CF₃ | H | CF₃CH₂ | SO₂ | 124-127° C. |
| c-6 | CF₃ | H | CF₃CH₂ | S | 215-216° C. |
| c-7 | CF₃ | H | ⁱPr | SO₂ | 223-226° C. |
| c-8 | CF₃ | H | ⁱPr | S | 200-202° C. |
| c-9 | CF₃ | H | Et | SO₂ | 213-214° C. |
| c-10 | CF₃ | H | Et | S | 169-173° C. |
| c-11 | CF₃ | H | ^{t}Bu | SO₂ | * |
| c-12 | CF₃ | H | ^{t}Bu | S | * |
| c-13 | CF₃ | H | ^{c}Pr | SO₂ | 208-209° C. |
| c-14 | CF₃ | H | ^{c}Pr | S | * |
| c-15 | CF₃ | H | MeOCH₂CH₂ | SO₂ | 182-184° C. |
| c-16 | CF₃ | H | MeOCH₂CH₂ | S | 164-165° C. |
| c-17 | CF₃ | H | ⁿPr | SO₂ | * |
| c-18 | CF₃ | H | ⁿPr | S | 112-116° C. |
| c-19 | CF₃ | CO₂Me | Me | SO₂ | 233-235° C. |
| c-20 | CF₃ | C(═O)Ph | Me | S | 198-199° C. |
| c-21 | CF₃ | Ac | Me | SO₂ | * |
| c-22 | CF₃ | C(═O)(4-ClPh) | Me | SO₂ | 235-238° C. |
| c-23 | CF₃ | C(═O)(4-MePh) | Me | SO₂ | 239-240° C. |
| c-24 | CF₃ | C(═O)(4-MeOPh) | Me | SO₂ | * |

Among the compounds described in Table 4, the compounds with * marked in the column of melting point were amorphous or viscous oil. The ¹H-NMR data thereof are shown in Table 5.

TABLE 5

| Compound | |
|---|---|
| number | ¹H-NMR data |
| c-11 | ¹H NMR (400 MHz, DMSO-d6): δ 1.70 (s, 9H), 2.39 |
| | (m, 2H), 3.22 (m, 2H), 3.67 (m, 2H), 7.91 (d, 1H), 8.06 |
| | (d, 1H), 11.33 (bs, 1H). |
| c-12 | ¹H NMR (400 MHz, DMSO-d6): δ 1.70 (s, 9H), 2.06 |
| | (m, 2H), 2.94 (m, 2H), 3.10 (m, 2H), 7.37 (d, 1H), 7.73 |
| | (d, 1H). |
| c-14 | ¹H NMR (400 MHz, DMSO-d6): δ 1.17-1.89 (m, 4H), |
| | 2.06 (m, 2H), 2.95 (m, 2H), 3.10 (m, 2H), 3.72 (m, 1H), |
| | 7.45 (d, 1H), 7.71 (d, 1H). |
| c-17 | ¹H NMR (400 MHz, DMSO-d6): δ 0.90 (t, 3H), 1.88 |
| | (m, 2H), 2.36 (m, 2H), 3.19 (m, 2H), 3.66 (m, 2H), |
| | 4.31 (t, 2H), 8.05 (bs, 2H), 11.7 (bs, 1H). |
| c-21 | ¹H NMR (400 MHz, CDCl3): δ 2.50 (s, 3H), 2.57 |
| | (m, 2H), 3.29 (m, 2H), 3.43 (m, 2H), 4.00 (s, 3H), 7.38 |
| | (d, 1H), 7.74 (d, 1H). |
| c-24 | ¹H NMR (400 MHz, CDCl3): δ 2.57 (m, 2H), 3.35 (m, |
| | 2H), 3.44 (m, 2H), 3.85 (s, 3H), 3.99 (s, 3H), 6.89 |
| | (d, 2H), 7.65 (d, 2H), 7.74 (d, 1H), 7.78 (d, 1H). |

### (Evaluation of Herbicidal Effect)

Next, it will be shown by the following Test Examples that the benzamide compound of the present invention is useful as an active ingredient of a herbicide.

### Test Example 1

### (1) Preparation of Emulsion for Testing

A POA allyl phenyl ether (4.1 parts by weight), a POE-POP glycol (1 part by weight), a POE sorbitan laurate (0.8 parts by weight), glycerin (2.6 parts by weight), dimethylformamide (65.9 parts by weight), N-methylpyrrolidone (5.1 parts by weight), cyclohexanone (15.4 parts by weight), and an aromatic hydrocarbon (5.1 parts by weight) were mixed and dissolved to prepare an emulsion. The compound of the present invention (4 mg) was dissolved in this emulsion (100 μL) to prepare an emulsion for testing. It is to be noted that POA means “polyoxyalkylene,” POE means “polyoxyethylene,” and POP means “polyoxypropylene.”

### (2) Spraying Treatment on Stems and Leaves

A 150 cm² pot was filled with soil, and seeds of Digitaria ciliaris and Matricaria chamomilla were sowed on the surface layer, and after the soil was covered lightly, the Digitaria ciliaris and Matricaria chamomilla were grown in a greenhouse. At the point in time when each plant was grown to a plant height of 2 to 4 cm, the emulsion for testing was diluted in such a way as to have a predetermined amount of an active ingredient and was sprayed on the leaves and stems in a spray amount of water of 250 L per hectare with a small-sized atomizer.

### (3) Evaluation

Two weeks or three weeks later, the weight of a living weed above the ground was measured for each weed in the nontreated zone and in the treated zone to calculate a herbicidal rate by the following calculation formula.

### (4) Calculation Formula for Herbicidal Rate

Herbicidal rate (%)=(weight of living weed above ground in nontreated zone−weight of living weed above ground in treated zone)/(weight of living weed above ground in nontreated zone)×100

The compounds shown in Table 6 were each sprayed in such a way that the spray amount was 250 g per hectare. As a result, all of the compounds exhibited a herbicidal activity of 80% or more to Digitaria ciliaris.

TABLE 6
Compound number

| | | | | |
|---|---|---|---|---|
| a-1 | a-23 | a-34 | b-1 | b-13 |
| a-3 | a-24 | a-35 | b-2 | b-14 |
| a-5 | a-25 | a-36 | b-5 | b-15 |
| a-6 | a-26 | a-37 | b-6 | b-16 |
| a-10 | a-27 | a-38 | b-7 | |
| a-11 | a-28 | a-43 | b-8 | |
| a-16 | a-29 | a-44 | b-9 | |
| a-17 | a-30 | a-45 | b-10 | |
| a-18 | a-31 | a-46 | b-11 | |
| a-22 | a-32 | a-47 | b-12 | |
| b-17 | c-2 | c-11 | c-20 | |
| b-18 | c-3 | c-12 | c-21 | |
| b-19 | c-4 | c-13 | c-22 | |
| b-20 | c-5 | c-14 | c-23 | |
| b-21 | c-6 | c-15 | c-24 | |
| b-22 | c-7 | c-16 | | |
| b-23 | c-8 | c-17 | | |
| b-24 | c-9 | c-18 | | |
| c-1 | c-10 | c-19 | | |

The compounds shown in Table 7 were each sprayed in such a way that the spray amount was 250 g per hectare. As a result, all of the compounds exhibited a weed-killing activity of 80% or more to Matricaria chamomilla.

TABLE 7
Compound number

| | | | |
|---|---|---|---|
| a-1 | a-21 | a-35 | b-5 |
| a-2 | a-22 | a-36 | b-6 |
| a-3 | a-23 | a-37 | b-7 |
| a-4 | a-26 | a-38 | b-8 |
| a-5 | a-27 | a-42 | b-10 |
| a-6 | a-28 | a-43 | b-11 |
| a-10 | a-29 | a-44 | b-12 |
| a-11 | a-31 | a-47 | b-13 |
| a-16 | a-33 | b-1 | |
| a-17 | a-34 | b-2 | |
| b-17 | c-2 | c-11 | c-20 |
| b-18 | c-3 | c-12 | c-21 |
| b-19 | c-4 | c-13 | c-22 |
| b-20 | c-5 | c-14 | c-23 |
| b-21 | c-6 | c-15 | c-24 |
| b-22 | c-7 | c-16 | |
| b-23 | c-8 | c-17 | |
| b-24 | c-9 | c-18 | |
| c-1 | c-10 | c-19 | |

### Test Example 2

### (1) Preparation of Emulsion for Testing

Emulsion for testing was prepared according to the preparation method described in Test Example 1.

### (2) Spraying Treatment on Stems and Leaves

A 150 cm² pot was filled with soil, and seeds of Avena sativa, Lolium multiflorum and Apera spica-venti were sowed on the surface layer, and after the soil was covered lightly, the Avena sativa, Lolium multiflorum and Apera spica-venti were grown in a greenhouse. At the point in time when Avena sativa was grown to a plant height of 16 to 20 cm, Lolium multiflorum was grown to a plant height of 10 to 14 cm, and Apera spica-venti was grown to a plant height of 2 to 4 cm, the emulsion for testing was diluted in such a way as to have a predetermined amount of an active ingredient and was sprayed on the leaves and stems in a spray amount of water of 250 L per hectare with a small-sized atomizer.

### (3) Evaluation

Three weeks later, the weight of a living weed above the ground was measured for each weed in the nontreated zone and in the treated zone to calculate a herbicidal rate by the following calculation formula.

### (4) Calculation Formula for Herbicidal Rate

Herbicidal rate (%)=(weight of living weed above ground in nontreated zone−weight of living weed above ground in treated zone)/(weight of living weed above ground in nontreated zone)×100

The compounds shown in Table 8 were each sprayed in such a way that the spray amount was 250 g per hectare. As a result, all of the compounds exhibited a herbicidal activity of 80% or more to Avena sativa.

TABLE 8
Compound number

| | | | | |
|---|---|---|---|---|
| a1 | a-29 | b-10 | c-2 | c-12 |
| a-3 | a-33 | b-11 | c-3 | c-13 |
| a-4 | a-35 | b-12 | c-4 | c-14 |
| a-11 | a-36 | b-13 | c-5 | c-15 |
| a-16 | a-37 | b-18 | c-6 | c-16 |
| a-18 | a-38 | b-19 | c-7 | c-17 |
| a-22 | a-43 | b-20 | c-8 | c-18 |
| a-26 | a-44 | b-21 | c-9 | c-22 |
| a-27 | b-6 | b-23 | c-10 | c-23 |
| a-28 | b-7 | b-24 | c-11 | c-24 |

Further, all of the compounds of compound numbers a-38, c-2, c-3, c-4, c-5, c-6, c-13, c-14, and c-15 exhibited a herbicidal activity of 80% or more with a spray amount of 64 g.

Further, all of the compounds of compound numbers a-38, c-5, and c-6 exhibited a herbicidal activity of 80% or more with a spray amount of 16 g.

The compounds shown in Table 9 were each sprayed in such a way that the spray amount was 250 g per hectare. As a result, all of the compounds exhibited a herbicidal activity of 80% or more to Lolium multiflorum.

TABLE 9
Compound number

| | | |
|---|---|---|
| a-3 | c-4 | c-15 |
| a-26 | c-5 | c-16 |
| a-35 | c-6 | c-17 |
| a-36 | c-7 | c-24 |
| a-37 | c-9 | |
| a-38 | c-10 | |
| b-6 | c-11 | |
| b-18 | c-12 | |
| b-20 | c-13 | |
| b-23 | c-14 | |

Further, all of the compounds of compound numbers a-38, c-6, c-14, c-15, and c-16 exhibited a herbicidal activity of 80% or more with a spray amount of 64 g.

The compounds shown in Table 10 were each sprayed in such a way that the spray amount was 250 g per hectare. As a result, all of the compounds exhibited a herbicidal activity of 80% or more to Apera spica-venti.

TABLE 10
Compound number

| | | | | |
|---|---|---|---|---|
| a-1 | a-29 | b-1 | b-21 | c-13 |
| a-3 | a-31 | b-6 | b-23 | c-14 |
| a-4 | a-33 | b-7 | b-24 | c-15 |
| a-5 | a-35 | b-11 | c-2 | c-16 |
| a-11 | a-36 | b-12 | c-5 | c-17 |
| a-22 | a-37 | b-13 | c-6 | c-18 |
| a-23 | a-38 | b-17 | c-8 | c-20 |
| a-26 | a-43 | b-18 | c-10 | c-21 |
| a-27 | a-44 | b-19 | c-11 | c-22 |
| a-28 | a-47 | b-20 | c-12 | c-24 |

Further, all of the compounds of compound numbers a-38, c-2, c-6, c-13, c-14, c-15, and c-16 exhibited a herbicidal activity of 80% or more with a spray amount of 64 g.

Further, the compound of compound number c-6 exhibited a herbicidal activity of 80% or more with a spray amount of 16 g.

All of the compounds randomly selected from among the benzamide compounds of the present invention exhibit the effect as described above, and therefore it can be understood that the benzamide compound of the present invention, including the compounds which cannot be given as examples, is a compound having a high herbicidal effect.

1. A compound of formula (II) or a salt thereof: 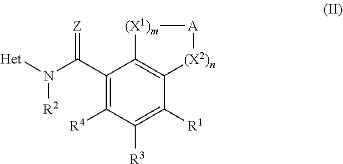
wherein
Het is a substituted or unsubstituted five-membered heteroaryl group;
R¹ is a substituted or unsubstituted C1-6 alkyl group, a substituted or unsubstituted C1-6 alkoxy group, a substituted or unsubstituted C1-6 alkylthio group, a substituted or unsubstituted C6-10 aryl group, a substituted or unsubstituted five- to six-membered heteroaryl group, or a halogeno group;
R² is a hydrogen atom, a substituted or unsubstituted C1-6 alkyl group, a substituted or unsubstituted C1-6 alkylcarbonyl group, a substituted or unsubstituted C1-6 alkoxycarbonyl group, or a substituted or unsubstituted C6-10 arylcarbonyl group;
X¹ is an oxygen atom or a sulfonyl group;
X² is an oxygen atom, a sulfenyl group, a sulfinyl group, a sulfonyl group, a group represented by —S(═NR)—, or a group represented by —S(═O)(═NR)—, wherein
each R independently is a hydrogen atom, a C1-6 alkyl group, a substituted or unsubstituted C1-6 alkylcarbonyl group, a substituted or unsubstituted C6-10 aryl group, or a cyano group;
m is 0 or 1, n is 0 or 1, and the sum of m and n is 1 or 2;
A is a substituted or unsubstituted C2-4 alkylene group, a substituted or unsubstituted C2-3 alkenylene group, or a substituted or unsubstituted C1-2 alkyleneoxy C1-C2 alkylene group;
R³ is a hydrogen atom, a substituted or unsubstituted C1-6 alkyl group, or a halogeno group;
R⁴ is a hydrogen atom, a substituted or unsubstituted C1-6 alkyl group, or a halogeno group; and
Z is an oxygen atom or a sulfur atom.

2. The compound or a salt thereof according to claim 1, wherein R³ is a hydrogen atom, R⁴ is a hydrogen atom, and Z is an oxygen atom.

3. The compound or a salt thereof according to claim 1, wherein m is 0, and n is 1.

4. A herbicide comprising as an active ingredient at least one compound or salt selected from the group consisting of the compound and a salt thereof according to claim 1.
